(19) United States
(12) Patent Application Publication
(10) Pub. No.: US 2024/0407930 A1
(43) Pub. Date: Dec. 12, 2024

(54) DESIGN AND METHOD FOR PRIMARY INTERVERTEBRAL DEVICE FIXATION AND REVISION SURGERY

(71) Applicants: Nebiyu Said OSMAN, Newington, CT (US); Jordan Andre BAUER, Woodbury, CT (US)

(72) Inventors: Nebiyu Said OSMAN, Newington, CT (US); Jordan Andre BAUER, Woodbury, CT (US)

(21) Appl. No.: 18/740,819

(22) Filed: Jun. 12, 2024

Related U.S. Application Data
(60) Provisional application No. 63/472,442, filed on Jun. 12, 2023.

(51) Int. Cl. A61F 2/44; A61B 17/04; A61F 2/30

(52) U.S. Cl. CPC A61F 2/447; A61B 17/0401; A61F 2002/30433; A61F 2002/30461; A61F 2002/30579; A61F 2002/30774; A61F 2002/30785

(57) ABSTRACT

An expandable intervertebral device and its fixation during spinal surgery features the following: The intervertebral device is stabilized using a latticework of high-strength suture that is either directly integrated or external to the device and has hollow channels for threading of high-strength suture, permitting direct integration of suture into the device for fixation via bone anchors. Expansion and retraction of the intervertebral device is achieved by modulating a dual-access screw, which can be accessed from either end of the intervertebral device. The versatility of the dual-access screw and easily detachable suture threads afford surgeons an ability to remove the intervertebral device from the most optimal direction during primary or revision surgery. This intervertebral device having the dual-access screw and high-strength suture fixation permits a secure, safe, versatile, and efficient approach to spinal surgery.

### CROSS REFERENCE TO RELATED APPLICATIONS

This application claims benefit to provisional application No. 63/472,442, filed 12 Jun. 2023, which is hereby incorporated by reference.

### BACKGROUND OF THE INVENTION

### Field of the Invention

The present invention relates to a method and apparatus for performing orthopaedic surgery; and more specifically relates to an implantable intervertebral device during used in spinal surgery.

### Description of Related Arts

The spine is a complex structure composed of many interconnected bony and soft-tissue components that permit dynamic movement, stability to the axial skeleton, and protection to the spinal cord. These components consist of a series of 33 vertebrae separated by intervertebral discs and connected by various ligamentous attachments that create the structure of the vertebral column. Most importantly, the spinal cord traverses through the spinal canal centrally within the vertebral column and extends from the medulla oblongata of the brainstem to the proximal lumbar region. The spinal cord is essential for transmitting and receiving motor and sensory signals to and from the peripheral nervous system.

Various spinal pathologies and disorders lead to disruption of the stability of the vertebral column and function of the spinal cord and nerve roots causing symptoms of pain, myelopathy, radiculopathy, and mild to severe dysfunction. Common spinal pathologies leading to these symptoms consist of degenerative disorders such as intervertebral disc herniation, spondylosis, spondylolysis, spondylolisthesis, spinal stenosis, foraminal stenosis, scoliosis, kyphosis, tumor, and other curvature abnormalities.

Management of spinal disorders can be challenging, e.g., including responding to different treatment modalities variably depending on patient specific factors and severity of the pathology. Symptoms and functions may improve with non-surgical management, which consists of medication and physical rehabilitation. However, failure with these conservative measures leaves surgical intervention as the next option. Addressing degenerative spinal disorders surgically are multifaceted and their application is reliant on the type and severity of the pathology. The different surgical interventions include laminectomy, discectomy, intervertebral fusion using static or dynamic device implantation with rods, fixation with buttress plates, bone fasteners, autografts, and osteoinductive allografts.

Specifically, with regards to pathologies resulting in degenerative disc disease, foraminal stenosis, central stenosis, facet arthropathy, dynamic instability of a motion segment and deformity, intervertebral device implantation is a mainstay surgical intervention. These implants can be leveraged to achieve different goals via different mechanisms depending on the clinical context including indirect decompression of neural elements, reduction of spondylolisthesis, stabilization of the motion segment, realignment of sagittal and coronal deformity, and interbody fusion among others. Previously, these devices were statics implants, however, recent advances in technology have introduced dynamically expandable intervertebral devices that are implanted in a condensed state and subsequently expanded into dimensions not afforded to their predecessors. Despite the increased use of these devices and improvement in technology, there are still significant deficiencies and complications related to intervertebral devices.

Migration and subsistence are two important postoperative complications of intervertebral devices that lead to adverse symptoms, dysfunction, pseudoarthrosis, damage to surrounding structures, failed fusion, and revision surgery. Previous literature reviewed by the inventors demonstrated that the main indications for revision surgery were related to symptomatic implant subsidence and migration. See references 1-5 listed below. Intervertebral device migration and subsidence have been shown to be influenced by multiple factors including endplate injury, pear-shaped implants, screw loosening, implant loosening, poor bone quality, adequacy of decompression, posterior positioning of an implant, unilateral placement of an implant, implant size, and removal of the anterior and posterior longitudinal ligaments. See references 2-3 and 6-11 listed below. Furthermore, intervertebral implant migration can lead to severe complications. In three case studies reviewed, patients suffered esophageal perforation and pharyngeal infection secondary to anterior migration of ACDF implants. See references 12-14 listed below. These complications are related to known fixation strategies prominently positioning cervical implants anteriorly that can irritate surrounding structures, such as the esophagus during ACDF. Furthermore, known cage designs lack multiple access points for expanding and collapsing spinal implants for removal via different approaches. Ultimately, the known intervertebral devices offered on the market are not fully optimized to decrease and prevent these adverse events.

The inventors searched and reviewed the database of the US Patent and Trademark Office and found other intervertebral devices that claim to expand and provide adequate spacing and fixation between one or more intervertebral spaces. For example, see the following U.S. Patents and patent applications: U.S. Pat. Nos. 11,596,522 B2, 11,596,523 B2, US 2023/0043823 A1, US 2023/0039330 A1,U.S. Pat. No. 11,564,724 B2, US 2023/0000638 A1, U.S. Pat. No. 11,529,242 B2, US 2022/0387184 A1, U.S. Pat. Nos. 11,517,444 B2, 11,602,439 B2, 11,612,499 B2). All of these patents or patent applications claim to disclose devices having either have properties related to an expandable intervertebral device that is either fixed with or without screws. However, none of these patents or patent applications disclose an expandable intervertebral device that is modulated via a dual-access screw with hollow channels for high-strength suture to either internally or externally fix and support the device to prevent migration and iatrogenic injury.

### SUMMARY OF THE INVENTION

This present invention describes a new and unique apparatus featuring an expandable intervertebral device and a method for fixation of the device during spinal surgery. In practice, this expandable intervertebral device is implanted in its retracted or condensed configuration between two vertebral bodies and subsequently expanded to reestablish the native intervertebral space and angle between the two vertebral bodies. This expandable intervertebral device include two central components and two plates (superior and inferior). The two central components have male and female parts that slide or glide over each other. These two components also have a transverse aperture with threads for a screw to be housed. The sliding or gliding movement between these two central components is modulated via a dual-access screw housed within the transverse aperture that connects the two central components. This dual-access screw is also the primary facilitator of expansion and retraction of this device. This expandable intervertebral device expands vertically via a superior and inferior plate. The two plates expand vertically in the opposite direction relative to each other and are modulated in relation to the central components. Pre-molded angled interlocks on the central components and the superior and inferior plates afford this device to act as one solid structure that has components that glide against each other to achieve expansion and retraction. Inclination is pre-set by the angle of the pre-molded interlocking trajectory between the plates and the central components.

Furthermore, this expandable intervertebral device has hollow channels in the superior and inferior plates that allows for high-strength suture to be threaded through for direct fixation into adjacent osseus structures via suture anchors. Additionally, high-strength suture can be used without threading through the device and fixed to adjacent osseus structures to create a low-profile lattice work that functions as a tension band and supports the intervertebral device externally. The benefit of this design is that the sutures can be easily released from varying angles and approaches to free the implants for removal, the suture can be grabbed to extract the implant in certain situations, and the use of suture allows for placement of fixation in the most optimal locations. This design affords surgeons the ability to account for each patient's unique anatomy and adjust the amount of tension to precisely correct deformities and avoid compressing the cage, which increases the risk of subsidence.

The expandable intervertebral device according to the present invention is different from all other known devices and systems described above, because its expansion and retraction are modulated via a dual-access screw, and it has hollow channels within it that affords direct or indirect fixation via high-strength suture and bone anchors. Since this expandable intervertebral device is modulated via the dual-access screw and is further supported via the high-strength suture, it will allow surgeons to maintain the use of their preferred surgical approach with the added benefit of a low-profile fixation method that inhibits device migration. Additionally, the dual-access screw and easily detachable suture afford the versatility during revision surgery to use a different approach and avoid scar tissue that complicates dissection and implant removal.

### SPECIFIC EMBODIMENTS

According to some embodiment, the present invention may take the form of an expandable intervertebral device, e.g., for implantation between two vertebral bodies, featuring a central component that consists of two pieces, a dual-access screw, and two plates.

The central component may include, or be configured with two pieces, with male and female parts that slide transversely over each other, and each has a centered aperture for a dual access screw to connect these two pieces.

The dual-access screw may be configured as a screw that traverses though the communicating apertures in the two pieces of the central component to connect them. This screw has dual-access, meaning a screwdriver can access and turn either end of the screw. One end is headless and one end is headed. The headed end of the screw is physically in contact internally with one piece of the central component to facilitate expansion or retraction of the device with each subsequent revolution of the screw.

The two plates may include, or be configured with, a superior and inferior plate that are connected to the central component via molded angled interlocks that affords dynamic sliding and expansion of the device facilitated by the dual-access screw, and hollow channels for high-strength suture to be threaded through and afford either direct or indirect fixation and support of the entire intervertebral device.

The expandable intervertebral device may include one or more of the following features presented:

The expandable intervertebral device may have a single or multiple hollow channels for high-strength suture to be threaded through.

The expandable intervertebral device may have one or multiple molded attachment sites that connect to a device inserter for implantation.

The expandable intervertebral device may be implanted in multiple orientations depending on the approach and device inserter attachment site used.

The expandable intervertebral device may be made partially or completely with radiolucent material.

### The Method

According to some embodiment, the present invention may take the form of a method for implantation, expansion, and fixation of an expandable]

intervertebral device, e.g., featuring steps of:
a. dissecting to one or more intervertebral space(s) with significant pathology, debriding surrounding joint space(s), and preparing the vertebral bodies surfaces;
b. fixing bone anchors with high-strength suture to the surrounding osseus structures of the vertebral column;
c. threading free-ends of high-strength suture through hollow channels in the expandable intervertebral device;
d. connecting a cannulated implant inserter to the expandable intervertebral device and implanting it between two vertebral bodies intraoperatively;
e. using an elongated screwdriver that slides through the cannulated implant inserter to contact one of the ends of the dual-access screw to expand the device;
f. injecting bone grafting material through the cannulated implant inserter that fills the surrounding openings in the intervertebral space and facilitates bony unionization;
g. fixing each of the free ends of the high-strength suture threaded through the expandable intervertebral device into surrounding osseus structures in a variety of latticework patterns for support;
h. fixing high-strength suture external to the expandable intervertebral device into surrounding osseus structures in a variety of latticework patterns for indirect support and buttressing of the device and functioning as a tension band to compress to the two endplates to the cage; and
i. removing the expandable intervertebral device in the setting of revision surgery via the same or different approach as the primary surgery afforded by the easily cut sutures and versatile access of the dual-access screw.

### FURTHER EMBODIMENTS

### An Expandable Intervertebral Device

The present invention may take the form of an expandable intervertebral device for insertion between two vertebral bodies to reestablish the appropriate space and angle of the spine, featuring a central component, a superior plate, an inferior plate and a dual-access screw.

The central component may include proximal and distal central pieces with superior molded angled interlocks and also with inferior molded angled interlocks, the proximal and distal central pieces being configured to slide transversely relative to each other.

The superior plate may include proximal and distal ends with molded angled interlocks configured to connect to the superior molded angled interlocks of the proximal and distal central pieces and allow for uninhibited sliding of the superior plate relative to the proximal and distal central pieces.

The inferior plate may include corresponding proximal and distal ends with corresponding molded angled interlocks configured to connect to the inferior molded angled interlocks of the proximal and distal central pieces and allow for uninhibited sliding of the inferior plate relative to the proximal and distal central pieces.

The dual-access screw may be configured to connect the proximal and distal central pieces and modulate transverse compression and expansion of the proximal and distal central pieces and vertical expansion and compression of the superior plate and the inferior plate respectively.

The expandable intervertebral device may include one or more of the following features:

The expandable intervertebral device may include one or more cannulated channels configured to pass through the expandable intervertebral device and receive high-strength suture to be threated through and subsequent fixation to adjacent osseus structures via bone anchors.

The proximal and distal central pieces may each have a respective cannulated opening configured for the dual-access screw to be threaded through.

The dual-access screw may include a head; and the respective cannulated opening in a proximal central piece of the proximal and distal central pieces has a diameter that is greater than a corresponding diameter where the head of the dual-access screw sits.

The dual-access screw may include a headless portion and a body having threads with an inner and outer diameter; and a distal central piece of the proximal and distal central pieces having a cannulated portion that is threaded and matches the inner and outer diameter of the threads of the body of the dual-access screw.

The dual-access screw may include a proximal end and a headless distal end, and the proximal end has a proximal head.

### Method for Inserting the Expandable Intervertebral Device Between Two Vertebral Bodies

By way of further example, the present invention may also include, or take the form of, a method for fixation and removal of the expandable intervertebral device according to claim 1, wherein the method comprises:
modulating with the dual-access screw vertical expansion and compression of the superior plate and the inferior plate by either transverse compression or expansion of proximal and distal central pieces respectively;
integrating the one or more cannulated channels within the expandable intervertebral device with one or more high-strength sutures for direct fixation via the one or more associated bone anchors; and
fixing externally the one or more high-strength sutures with the one or more associated bone anchors to surrounding osseus structures for indirect buttressing of the expandable intervertebral device

The method may also include one or more of the following features:

The method may include rotating a proximal head of a proximal end of the dual-access screw clockwise, or a headless distal end counterclockwise for vertical expansion of the superior plate and the inferior plate.

The method may include rotating a proximal head of a proximal end of the dual-access screw counterclockwise, or rotating a headless distal end clockwise for vertical compression of the superior plate and the inferior plate.

The method may include fixing the one or more high-strength sutures at either one or more ends of the expandable intervertebral device.

The method may include either integrating the one or more high-strength sutures into the one or more cannulated channels of the expandable intervertebral device, or using the one or more high-strength sutures external to the expandable intervertebral device as a low-profile buttress.

The method may include fixing the one or more high-strength sutures via the one or more associated bone anchors in vertical and/or crossing latticework patterns.

The method may include adapting the expandable intervertebral device to an insertion handle for implantation and subsequent bone graft integration for intervertebral unionization.

The method may be implemented in a revision surgery using a similar or different approach as is used in a primary surgery by exposing ends of the expandable intervertebral device for access to one of a proximal or distal end of the dual-access screw for removal of the expandable intervertebral device and insertion of a new intervertebral implant.

The method may include configuring each of the proximal and distal central pieces with a respective cannulated opening for threading the dual-access screw through.

By way of still further example, and according to some still further embodiments, the present invention may take the form of a method for inserting an expandable intervertebral device between two vertebral bodies to reestablish the appropriate space and angle of the spine, featuring:
configuring a central component having proximal and distal central pieces with superior molded angled interlocks and also with inferior molded angled interlocks to slide transversely relative to each other;
configuring a superior plate having proximal and distal ends with molded angled interlocks to connect to the superior molded angled interlocks of the proximal and distal central pieces and allow for uninhibited sliding of the superior plate relative to the proximal and distal central pieces;
configuring an inferior plate having corresponding proximal and distal ends with corresponding molded angled interlocks to connect to the inferior molded angled interlocks of the proximal and distal central pieces and allow for uninhibited sliding of the inferior plate relative to the proximal and distal central pieces; and
connecting with a dual-access screw the proximal and distal central pieces and modulating transverse compression and expansion of the proximal and distal central pieces and vertical expansion and compression of the superior plate and the inferior plate respectively.

The method may also include one or more of the features set form above, e.g., including configuring one or more cannulated channels to pass through the expandable intervertebral device and receive one or more high-strength sutures to be threated through for subsequent fixation to adjacent osseus structures via one or more associated bone anchors.

### BRIEF DESCRIPTION OF DRAWING

The drawing include FIGS. 1-19, as follows:

FIG. 1 is a perspective view of a proximal end of an expandable intervertebral device in a condensed configuration according to some embodiments of the present invention, e.g., that includes hollow channels for threading of a high-strength suture, a headed end of a dual-access screw for expansion and retraction modulation, and attachment sites for a device inserter.

FIG. 2 is a perspective view of the proximal end of the expandable intervertebral device in an expanded configuration according to some embodiments of the present invention, e.g., including superior and inferior plates that expand vertically as central components condense transversely.

FIG. 3 is a perspective view of a distal end of the expandable intervertebral device in the condensed configuration shown in FIG. 1 according to some embodiments of the present invention, e.g., that includes the hollow channels for threading of the high-strength suture and an aperture for a headless end of the dual-access screw.

FIG. 4 is a perspective view of the distal end of the expandable intervertebral device in the expanded configuration shown in FIG. 2 according to some embodiments of the present invention, e.g., where the superior and inferior plates expand vertically as the central components condense transversely and include a headless end of the dual-access screw for modulation of cage expansion and retraction.

FIG. 5 is a perspective view of the proximal end of the two central components in the condensed configuration shown in FIG. 1 according to some embodiments of the present invention, e.g., including the dual-access screw for expansion and retraction modulation and molded angled interlocks on proximal and distal components that link to the superior and inferior plates and allow sliding motion.

FIG. 6 is a perspective view of the proximal end of the two central components in the expanded configuration shown in FIG. 2 according to some embodiments of the present invention, e.g., including the dual-access screw for expansion and retraction modulation and molded angled interlocks on both the proximal and distal components that link to the superior and inferior plates and allow sliding motion.

FIG. 7 is a perspective view of the distal end of the two central components in the condensed configuration shown in FIG. 3 according to some embodiments of the present invention, e.g., including the dual-access screw for expansion and retraction modulation and molded angled interlocks on both the proximal and distal components that link to the superior and inferior plates and allow sliding motion.

FIG. 8 is a perspective view of the distal end of the two central components in the condensed configuration shown in FIG. 4 according to some embodiments of the present invention, e.g., including the dual-access screw for expansion and retraction modulation and molded angled interlocks on both the proximal and distal components that link to the superior and inferior plates and allow sliding motion.

FIG. 9 is an exploded view of the distal ends of the two central components and dual-access screw shown in FIGS. 3, 4, 7 and 8 according to some embodiments of the present invention, e.g., including FIG. 9A showing the hollow aperture in the proximal component, FIG. 9B showing the hollow shaft in the distal component that connects to the proximal component, and FIG. 9C showing the dual-access screw with the headed proximal end and headless distal end.

FIG. 10 is an exploded view of the proximal ends of the two central components and dual-access screw shown in FIGS. 1, 2, 5 and 6 according to some embodiments of the present invention, e.g., including FIG. 10A showing the dual-access screw with the headed proximal end and headless distal end, FIG. 10B showing the hollow aperture with screw threads in the distal component, and FIG. 10C showing the proximal end of the hollow aperture of the proximal component where the headed end of the screw rotates.

FIG. 11 is a lateral view of the expandable intervertebral device in the condensed configuration according to some embodiments of the present invention, e.g., including illustration of one configuration for hollow channels within the cage that high-strength suture can be threaded through; and bone anchors and locking devices shown for method of fixation.

FIG. 12 is a lateral view of implantation of the expandable intervertebral device in the condensed configuration during single level lumbar spinal surgery in accordance with the design and method of the present invention, and includes FIG. 12A showing an expanded view of the expandable intervertebral device in the condensed configuration during the single level lumbar spinal surgery.

FIG. 13 is a lateral view of expansion of the implanted expandable intervertebral device during single level lumbar spinal surgery in accordance with the design and method of the present invention, and includes FIG. 13A showing an expanded view of the expandable intervertebral device during the single level lumbar spinal surgery.

FIG. 14 is a lateral view of fixation of the expanded intervertebral device with integrated high-strength suture via bone anchors during single level lumbar spinal surgery, and includes FIG. 14A showing an expanded view of the expandable intervertebral device during the single level lumbar spinal surgery.

FIG. 15 is an anterior view of the expandable intervertebral device with integrated high-strength suture fixed to adjacent vertebral bodies via bone anchors during single level cervical spine surgery according to the method and design of the present disclosure, e.g., one possible high-strength suture latticework configuration.

FIG. 16 is an anterior view of the expandable intervertebral device with integrated high-strength suture fixed to adjacent vertebral bodies via bone anchors during single level cervical spine surgery according to the method and design of the present invention, e.g., another unique high-strength suture latticework configuration.

FIG. 17 is an anterior view of the expandable intervertebral device with integrated high-strength suture fixed to adjacent vertebral bodies via bone anchors during single level cervical spine surgery according to the method and design of the present invention, e.g., another unique high-strength suture latticework configuration.

FIG. 18 is an anterior view of the expandable intervertebral device with external high-strength suture fixed to adjacent vertebral bodies via bone anchors during single level cervical spine surgery according to the method and design of the present invention, e.g., another unique high-strength suture latticework configuration.

FIG. 19 is an anterior view of the expandable intervertebral device with external high-strength suture fixed to adjacent vertebral bodies via bone anchors during multi-level cervical spine surgery according to the method and design of the present invention, e.g., another unique high-strength suture latticework configuration.

### DETAILED DESCRIPTION OF VARIOUS EMBODIMENTS

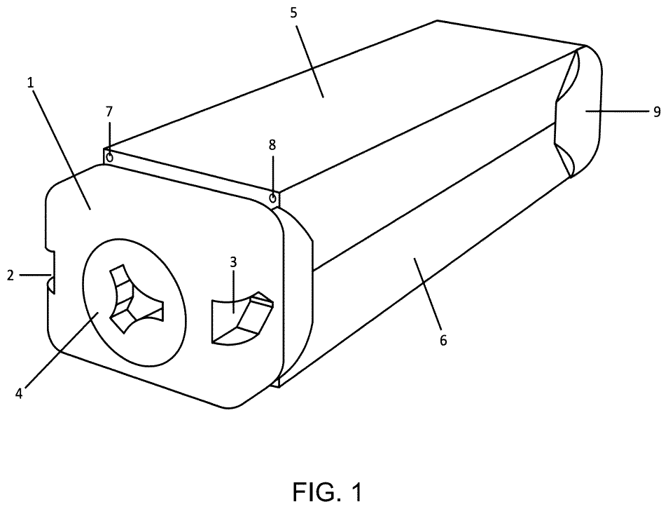

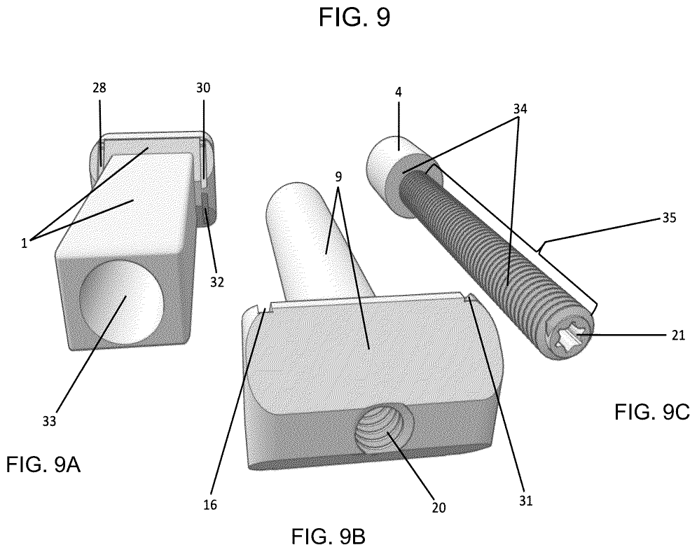

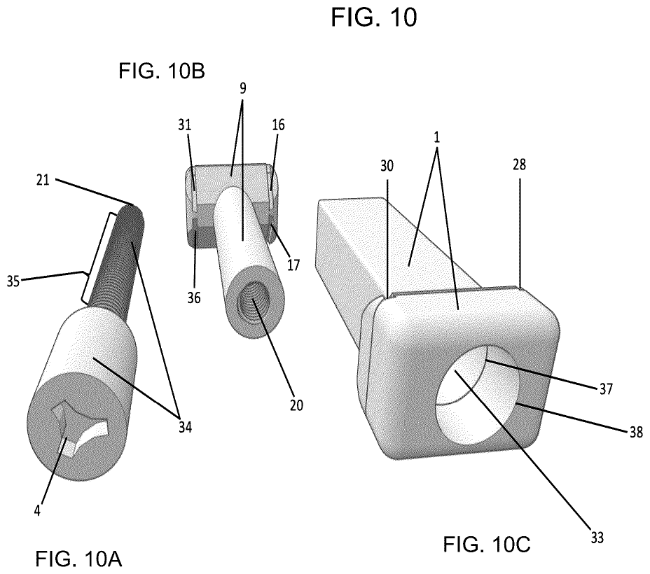

FIG. 1 shows a new and unique expandable intervertebral device according to some embodiments of the present invention, e.g., that includes hollow channels (7, 8) for high-strength suture to be threaded through for secure fixation to prevent device migration. The vertical expansion and retraction of superior and inferior plates (5, 6) is modulated by the extension and compression of the proximal and distal central pieces or components (1, 9) controlled via a dual-access screw ((34), see FIGS. 9C and 10A) with a proximal headed end (4) and distal headless end.

In particular, FIG. 1 shows a proximal end of the expandable intervertebral device in a condensed configuration. A proximal central component (1) has attachment sites (2, 3) for a device inserter to connect to; it has a hollow aperture where the proximal headed end (4) of the dual-access screw ((34), see FIGS. 9C and 10A) sits and freely rotates; and it has a hollow tube that extends distally (not seen in this Figure) and slides over part of a distal central component (9). The superior plate (5) sits on top of the proximal and distal central components (1, 9) and connects to them via molded angled interlocks (not see in this Figure) that allows for free sliding. The inferior plate (6) sits on the bottom of the proximal and distal central components (1, 9) and connects to them via molded angled interlocks (not seen in this Figure) that allows for free sliding.

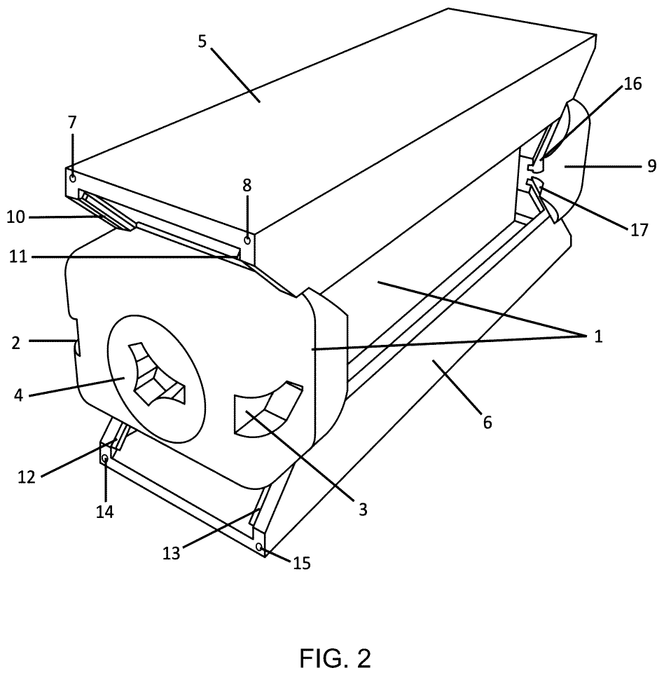

FIG. 2 shows the proximal end of the expandable intervertebral device shown in FIG. 1 in an expanded configuration. The two central components (1, 9) are compressed together resulting in a vertical expansion of the superior and inferior plates (5, 6). The central components (1, 9) are connected to the superior and inferior plates (5, 6) via sixteen molded angled interlocks. The proximal central component (1) has four molded angled interlocks (not shown) that are associated with two molded angled interlocks (10, 11) on the superior plate (5) and two molded angled interlocks (12, 13) on the inferior plate (6). The distal central component (9) has four molded angled interlocks (16, 17, the other two not shown) that are associated with two molded angled interlocks (not shown) on the superior plate (5) and two molded angled interlocks (not shown) on the inferior plate (6).

This interlocking connection between the superior and inferior plates (5, 6) with the two central components (1, 9) allows for device expansion via compression of the two central components (1, 9) regulated by rotating the proximal head end (4) of the dual-access screw ((34), see FIGS. 9C and 10A) clockwise. Device retraction is achieved via counterclockwise rotation of the head (4) of the dual-access screw (34) or clockwise rotation of the headless end of the dual-access screw (34) at the distal end not shown. Additionally, the hollow tube of the proximal central component (1) acting as the female part is shown enveloping the hollow tube, e.g., male part, of the distal central component (9)

The superior and inferior plates (5, 6) each have two hollow channels (7, 8, 14, 15) that extend transversely from the proximal face of the plates to the distal ends. These hollow channels (7, 8, 14,15) allow for high-strength suture to be threaded through the device for fixation during surgery. As claimed in this present invention this device can have one or multiple hollow channels for high-strength suture access through the plates (5, 6) or central components (1, 9).

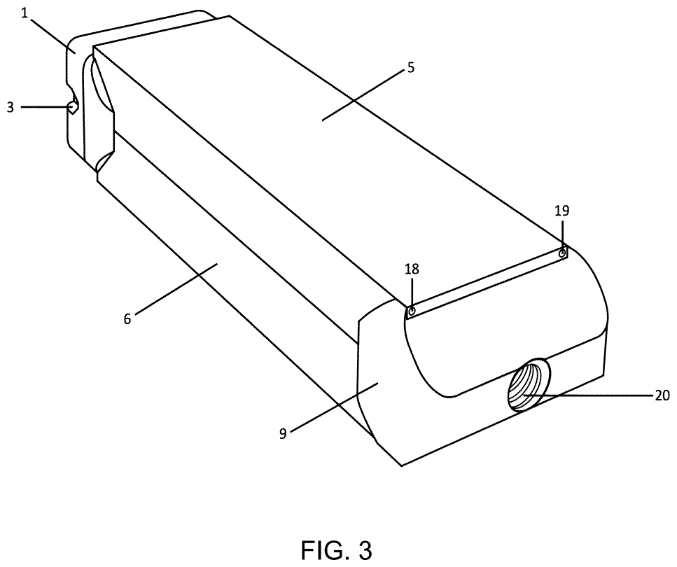

FIG. 3 shows a perspective view of the distal end of the expandable intervertebral device in the condensed configuration. The hollow channels (18, 19) at the distal end of the superior plate (5) communicate the openings (7, 8) at the proximal end of the plate. The distal central component (9) has a threaded aperture (20) that communicates with the proximal central component (1). The dual-access screw ((34), see FIGS. 9C and 10A) connects the two central components (1, 9) and the headless end of the dual-access screw (34) is visible and accessible at the end of the aperture (20) when the device is in the expanded configuration.

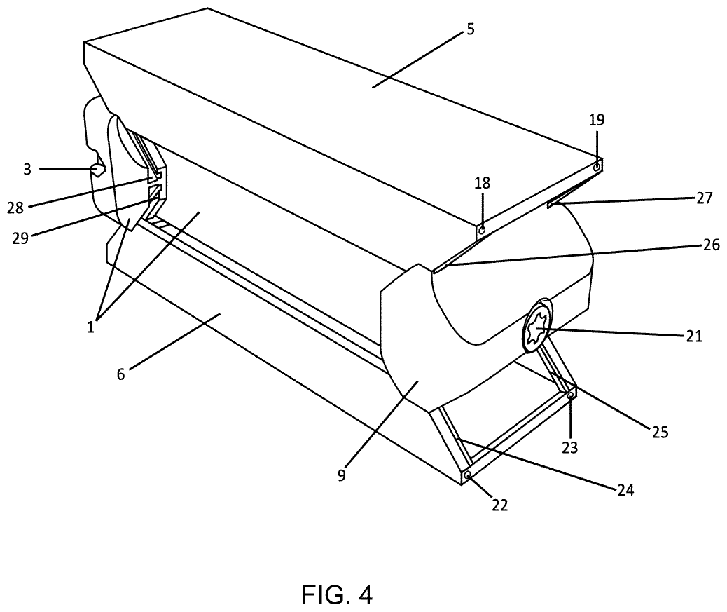

FIG. 4 shows a perspective view of the distal end of the expandable intervertebral device in the expanded configuration. The two central components (1, 9) are compressed together via the dual-access screw ((34), see FIGS. 9C and 10A) and associated molded angled interlocks resulting in vertical expansion of the superior and inferior plates (5,6). The headless portion (21) of the dual-access access screw (34) is visible in the device's expanded configuration. Application of clockwise rotation to the headless portion (21) of the dual-access screw (34) or counterclockwise rotation of the headed portion (4) would result in vertical compression of the superior and inferior plates (5, 6) and extension of the central components (1, 9).

From this view, two of the hollow channels (18, 19) in the superior plate (5) and two of the hollow channels (22, 23) in the inferior plate (6) are visible. The two channels (22, 23) in the distal end of the inferior plate (6) communicate with the openings (14, 15) proximally. As described, these hollow channels permit access for high-strength suture to be threaded through.

Two of the four molded angled interlocks (24, 25) are visible on the inferior plate (6), and two of the four molded angled interlocks (26, 27), are visible on the superior plate (5). The molded angled interlocks (24, 26) on the inferior and superior plates (5, 6) are associated with the molded angled interlocks (16, 17) on the distal central component shown in FIG. 2. The molded angle interlocks (28, 29) on the proximal central component are associated with the molded angled interlocks (11, 13) shown in FIG. 2.

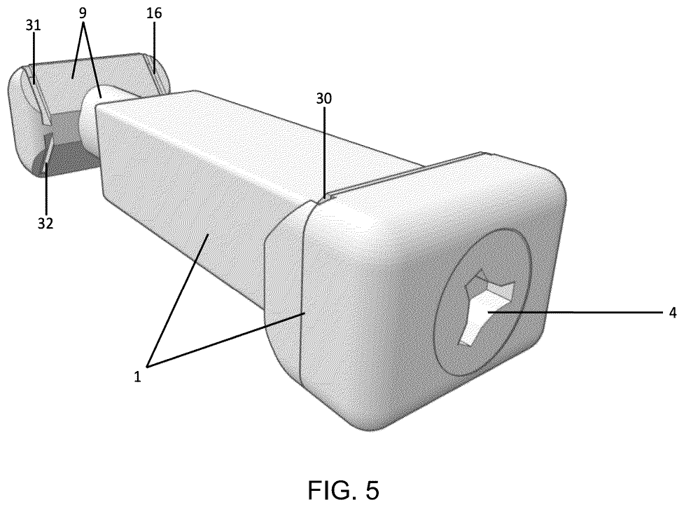

FIG. 5 is a perspective view of the proximal end of the expandable intervertebral device in the condensed configuration without the superior and inferior plates to better visualize the structure and mechanism of the two central components (1, 9). Specifically, the proximal central component (1) is a hollow tubular structure that houses the headed portion (4) of the dual-access screw ((34), see FIGS. 9C and 10A) most proximally, and as it continues distally the component acts as a female part that slides over the hollow tubular male part of the distal central component (9) permitting connection of the two parts via the dual-access screw (34). Furthermore, four molded angled interlocks are visible in this view. The one interlock (30) seen on the proximal central component is associated with an interlock (10) on the superior plate (5) shown in FIG. 2; and the three interlocks (16, 31, 32) on the distal central component (9) are associated with interlocks (25, 26, 27) on the superior and inferior plates (5, 6).

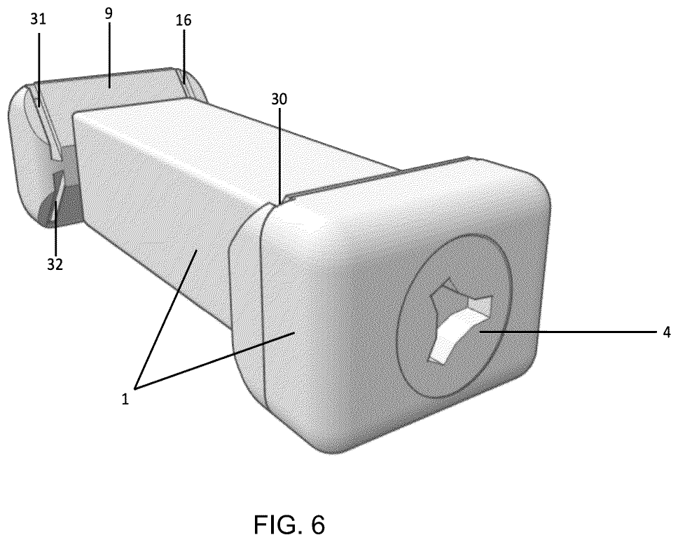

FIG. 6 is a perspective view of the proximal end of the expandable intervertebral device in the expanded configuration without the superior and inferior plates to better visualize the structure and mechanism of the two central components (1, 9). In this configuration and view, the female portion of the proximal central component (1) can be seen completely enveloping the male portion of the distal central component (9), which leads to vertical expansion of the superior and inferior plates not shown.

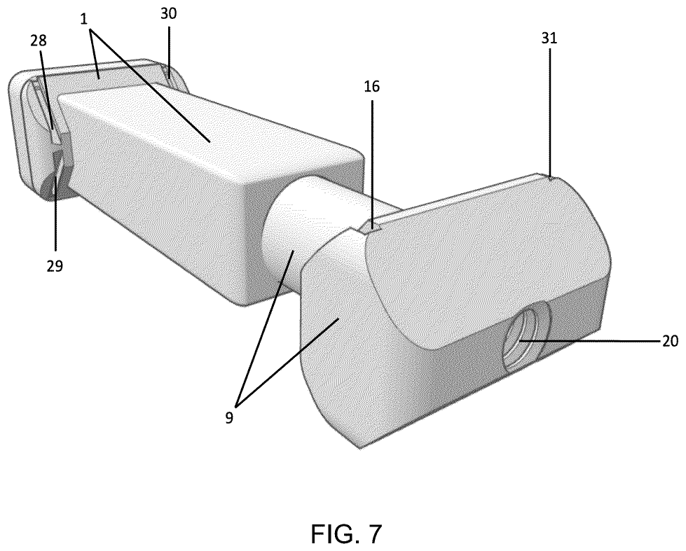

FIG. 7 is a perspective view of the distal end of the expandable intervertebral device in the condensed configuration without the superior and inferior plates to better visualize the structure and mechanism of the two central components (1, 9). Similarly, as described in FIG. 5, the proximal central component in a hollow tubular structure that acts as a female part enveloping the tubular male part of the distal central component (9). From this view the tubular male part of the distal central component (9) can be better visualized extending proximally through the hollow female part of the proximal central component (1). Centrally within the distal central component is a hollow tubular aperture (20) with threads for the distal end of the dual-access screw ((34), see FIGS. 9C and 10A) to traverse through during device expansion and contraction.

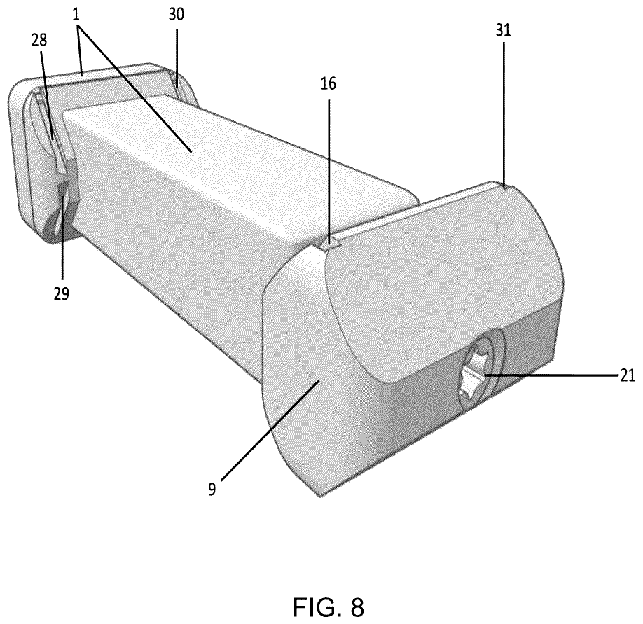

FIG. 8 is a perspective view of the distal end of the expandable intervertebral device in the expanded configuration without the superior and inferior plates to better visualize the structure and mechanism of the two central components (1, 9). In this configuration and view, the female portion of the proximal central component (1) can be seen completely enveloping the male portion of the distal central component (9), which leads to vertical expansion of the superior and inferior plates not shown. Furthermore, the headless portion of the dual-access screw ((34), see FIGS. 9C and 10A) is visualized at the end of the distal central component (9). Application of clockwise rotation to the headless end (21) of the dual access screw (34) will result in extension of the two central components (1, 9) and vertical compression of the superior and inferior plates not shown.

FIG. 9 includes exploded perspective views of the distal ends of the two central components (1, 9) shown in FIGS. 9A and 9B and the dual-access screw (34) shown in FIG. 9C. FIG. 9A shows the proximal central component (1) in isolation, which includes a hollow tubular female part that extends distally and has an aperture (33). The diameter of this hollowed aperture (33) is slightly larger in diameter compared to the outer diameter of the tubular male part of the distal central component (9) in FIG. 9B. This aspect allows for the distal central component (9) to slide transversely through the aperture (33) in the proximal central component (1). Three molded angled interlocks (28, 30, 32) in the proximal central component and two molded angled interlocks (16, 31) in the distal central component (9) are associated with their respective interlock connection on the superior and inferior plates (5,6) shown above.

The dual-access screw (34) shown in FIG. 9C is unique from all other screw types, because it has dual-access affording a screwdriver to interact with this screw from either end (4, 21). This dual-access screw (34) includes a headed proximal end (4) that sits within the proximal central component (1), a threaded body (35) that traverses through the apertures (33, 20) in the two central components (1, 9). The threads (35) of this dual-access screw (34) only interact with the threads within the threaded opening (20) of the distal central component (9). As set forth earlier, the aperture (33) in the proximal central component (1) has a diameter larger than the outer diameter of the tubular male part of the distal central component (9), and thus does not contact any threads and does not directly contact the body and threads (35) of the dual-access screw (34). Lastly, the dual-access screw's (34) has a distal end (21) that is headless, which allows for it to traverse through the threaded opening (20) of the distal central component (9) with the added benefit of versatile screwdriver access.

FIG. 10 shows the proximal ends of the two central components (1, 9) shown in FIGS. 10b and 10C and the dual-access screw (34) shown in FIG. 10A. The threads (35) on the body of the dual-access screw (34) in FIG. 10A fit to the threaded opening (20) in the distal central component (9) in FIG. 10B. The head (4) of the dual-access screw (34) in FIG. 10A fits within the proximal opening (33) of the proximal central component (1) in FIG. 10C. Specifically, the opening (33) contains two lips, one distal (37) and proximal aperture (38), that are slightly smaller in diameter compared to the outer diameter of the head (4) of the dual-access screw (34). This quality keeps the head of the screw (4) contained but permits free rotation and is essential to compression and expansion of the two central components (1, 9) relative to each other via the follow mechanism: For example, when the device is in the condensed state, the two central components (1, 9) are extended comparatively. When the head (4) of the dual-access screw (34) is rotated clockwise, the threads contact the threads of the opening (20) in the distal central component (9), and the head of the screw (4) contacts the lip (37) of the proximal central component (1) resulting in a compressing the two central components (1, 9) together. The equal but opposite result occurs when the device is in the expanded state and the dual-access screw (34) is rotated counterclockwise from the head of the screw (4) or clockwise from the headless end (21) of the screw (34), because the head of the screw (4) contacts the lip (38) and pushes or extends the two central components (1, 9) away from each other. The headed end of the screw (4) contacts the inner and outer lip (37, 38) of the proximal central component (1) to produce a condensed and retracted state respectively.

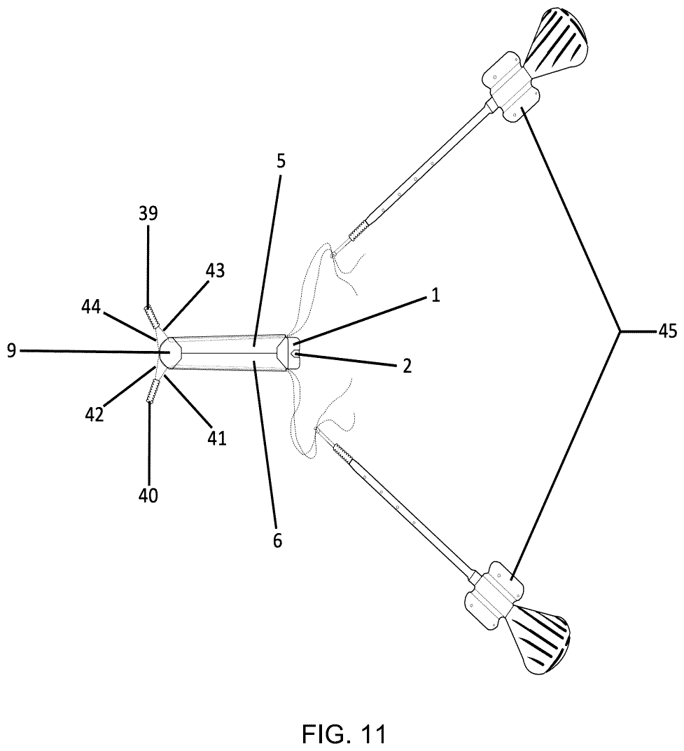

FIG. 11 shows the expandable intervertebral device in the condensed configuration with high-strength suture wire threaded through and integrated within the device. Two bone anchors (39, 40), each with two sutures (41, 42, 43, 44) emitting from them, are passed from the distal to proximal ends of the device using the hollow channel(s) as described above. The proximal ends of the sutures are not fixed and freely accessible for bone anchor inserting devices (45). By way of further example, the mechanisms and products used for bone anchor insertion and the high-strength suture can include the following from Arthrex: knotless anchors, knotted anchors, curved spears and flexible 1.6 mm drills, Swivelock device, FiberWire, FiberTak, FiberTape, and TigerTape.

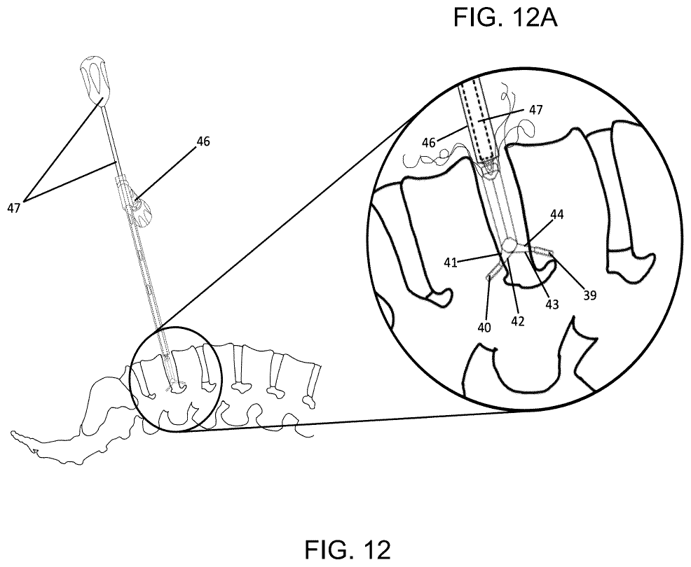

FIG. 12 shows the expandable intervertebral device being implanted in the condensed configuration within the L4-L5 intervertebral space during an anterior lumber interbody fusion (ALIF) surgery. By way of further example of devices used in the market, a cannulated device or implant inserter (46) is cannulated which affords access for an implant screwdriver (47) to contact the proximal end of the implant. This technology has been disclosed in in U.S. Pat. No. 11,554,025 B1. Bone anchors (39, 40) are seen fixed within the posterior portions of the L4 and L5 vertebral bodies. High-strength sutures (41, 42, 43, 44) emitting from the anchors (39, 40), are threaded through the device and the free ends of the sutures exist anterior to the vertebral bodies.

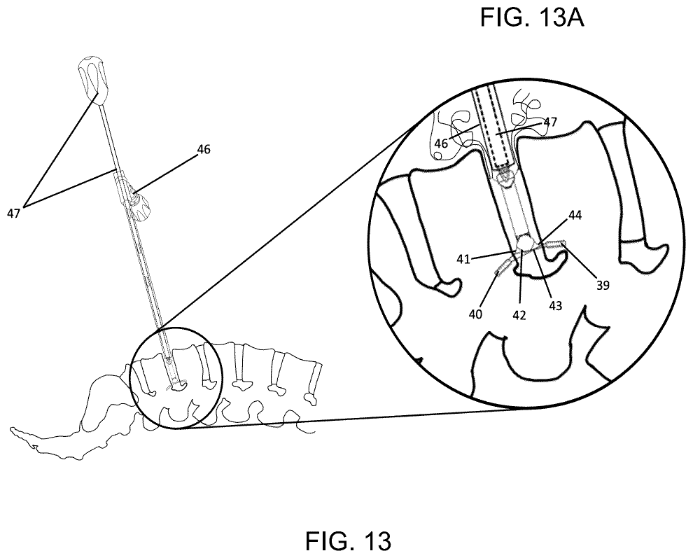

FIG. 13 shows the implanted expandable intervertebral device shown in FIG. 12 being expanded within the L4-L5 intervertebral space during the ALIF surgery. Once the implant screwdriver (47) is removed, a bone graft inserter can be placed through the cannulated opening in the cannulated device or implant inserter (46) to inject bone graft material and fill the open space within the intervertebral space.

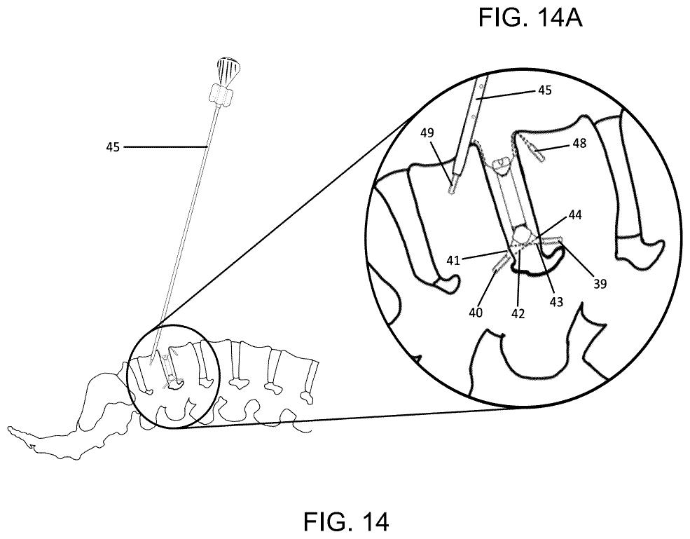

FIG. 14 shows the expandable intervertebral device in the expanded configuration shown in FIG. 13 being fixed with bone anchors (two posterior (39, 40) and two anterior (48, 49)), and high-strength suture (41, 42, 43, 44). The anterior bone anchor (49) is being placed into the L5 vertebral body by an existing device (45). An extended shaft on Arthrex's Swivelock device would be an appropriate modification for anchor fixation of this device during surgery.

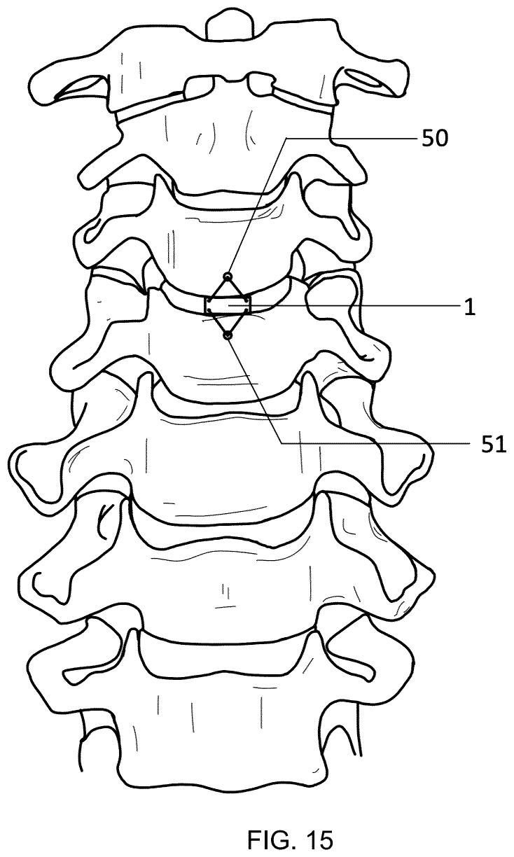

FIG. 15 shows a cervical spine having one expandable intervertebral device placed within the C3-C4 intervertebral space and one possible suture latticework configuration. This configuration consists of high-strength sutures integrated within the intervertebral device exiting anteriorly through the proximal central component (1). There are two sutures exiting the device superiorly that are fixed to a single bone anchor (50) placed superiorly in the body of C3, and two suture exiting inferiorly that are fixed to a single bone anchor placed inferiorly (51) in the body of C4.

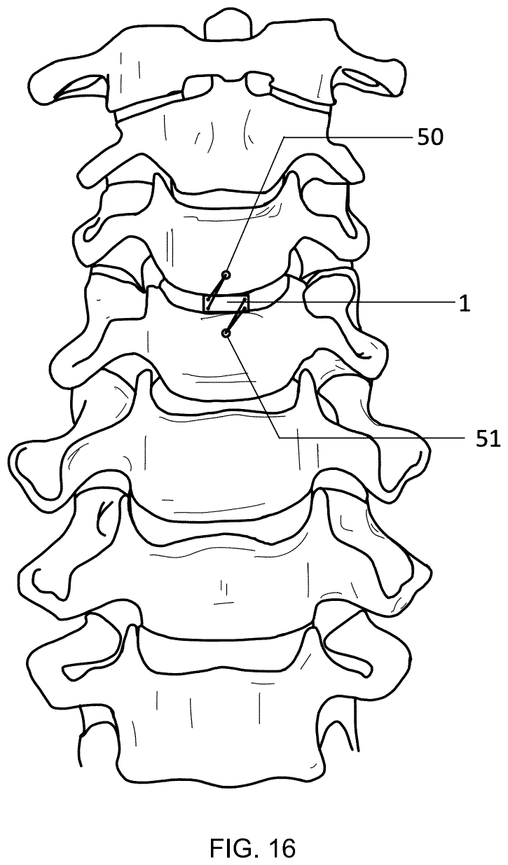

FIG. 16 shows a cervical spine having one expandable intervertebral device placed within the C3-C4 intervertebral space and one possible suture latticework configuration. This configuration consists of high-strength sutures integrated within the intervertebral device exiting anteriorly through the proximal central component (1). There are two sutures exiting the device superiorly and two sutures exiting inferiorly. The two sutures on the anatomic right side are fixed with a bone anchor (50) superiorly in the body of C3, and the two sutures on the anatomic left side are fixed with a bone anchor (51) inferiorly in the body of C4.

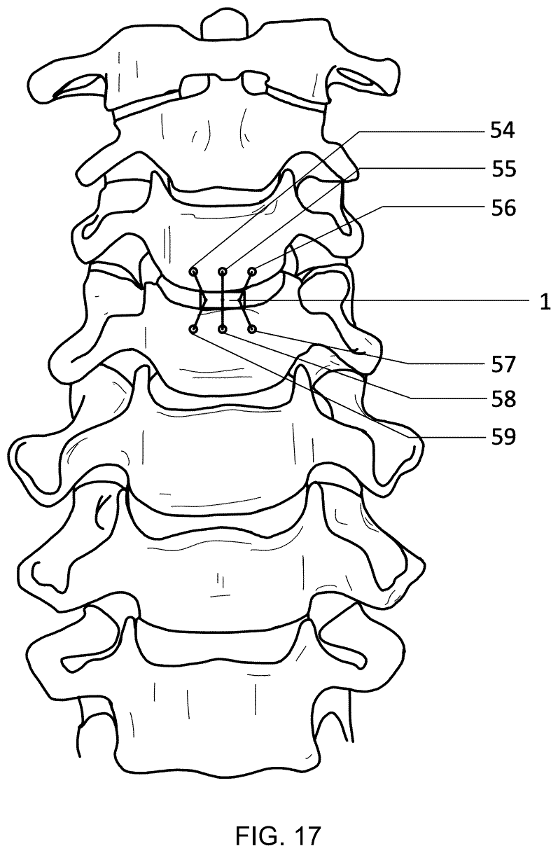

FIG. 17 shows a cervical spine having one expandable intervertebral device placed within the C3-C4 intervertebral space and one possible suture latticework configuration. This configuration consists of six high-strength sutures integrated within the intervertebral device exiting anteriorly through the proximal central component (1). There are three sets of two sutures exiting the device in a horizontal line with two sutures exiting on the left, right, and middle of the device. One sutures from each opening (three is total) are fixed either superiorly or inferiorly in the body of C3 and C4 with bone anchors (54, 55, 56, 57, 58, 59).

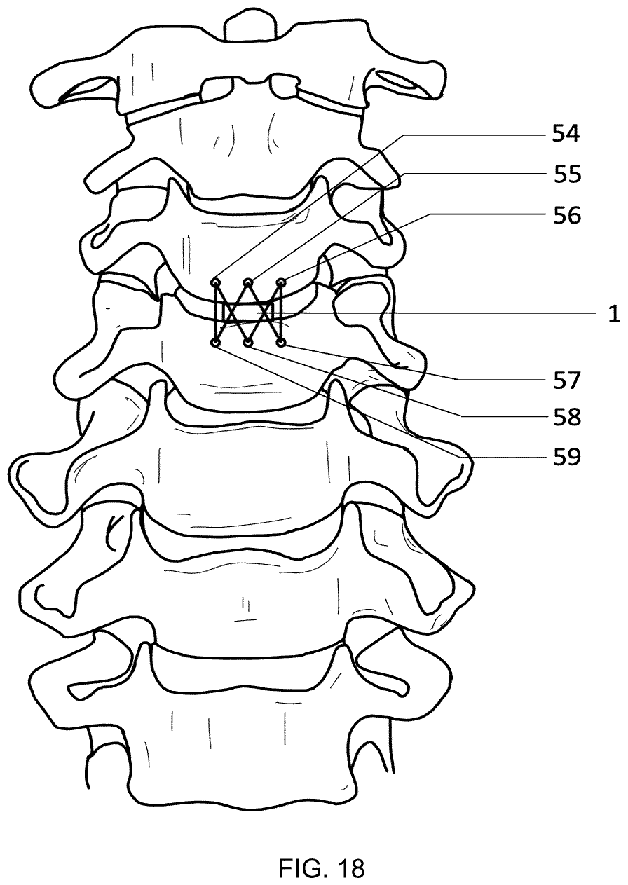

FIG. 18 shows a cervical spine having one expandable intervertebral device placed within the C3-C4 intervertebral space and one possible suture latticework configuration. This configuration consists of high-strength suture external to the device and interconnected via six total bone anchors (three superior and three inferior to the device). The superior bone anchors (54, 55, 56) are placed in the body of C3 and contain two sutures fixed to them. Subsequently, these sutures are passed inferiorly and fixed with anchors (57, 58, 59) in the body of C4 in a crossing pattern to externally buttress the device and provide compression when tensioned.

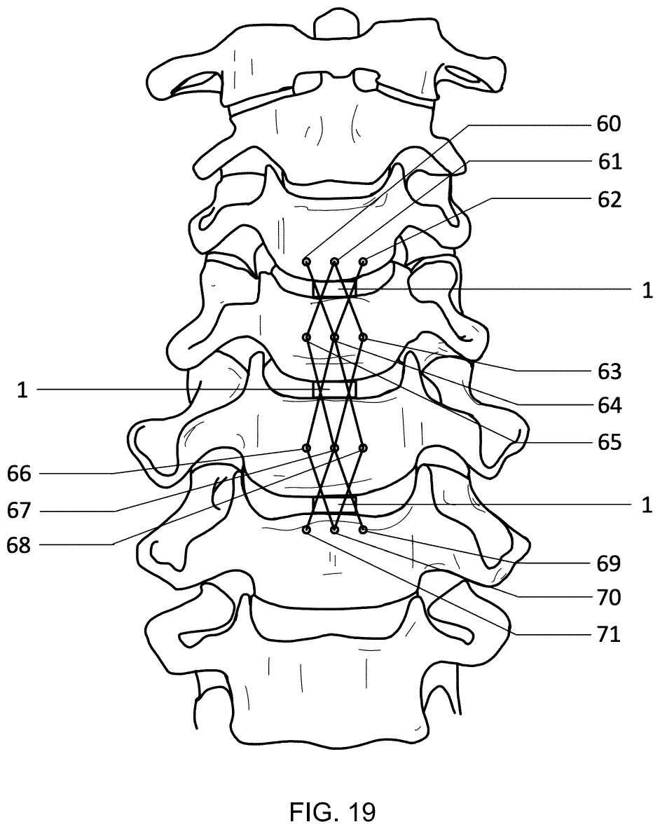

FIG. 19 shows a cervical spine with three intervertebral implants placed within the C3-C4, C4-C5, C5-C6 intervertebral spaces and one possible suture latticework configuration. This configuration consists of high-strength suture external to the devices and interconnected via twelve total bone anchors (e.g., three in the superior vertebral body and three in the inferior vertebral relative to each device). Each anchor (60, 61, 62, 53, 64, 65, 66, 67, 68, 69, 70, 71) has high-strength suture fixed within them and placed in a weaving latticework that buttresses each device externally.

In some of the embodiments set forth herein, the expandable intervertebral device and method can be implemented in various surgical approaches to the spine such as, anterior, posterior, posterior mid-line, direct lateral, posterolateral oblique, and anteriolateral oblique to complete procedures such as anterior lumbar interbody fusion (ALIF), oblique lumbar interbody fusion (OLIF), lateral lumbar interbody fusion (LLIF), transforaminal lumbar interbody fusion (TLIF), posterior lumbar interbody fusion (PLIF), anterior cervical discectomy and fusion (ACDF). The present disclosure can be employed with procedures treating any single or multilevel spine pathology and any region of the spinal column (lumbar, cervical, thoracic, sacral, or pelvic).

### REFERENCES

1. Chen, L., Yang, H., & Tang, T. (2005). Cage migration in spondylolisthesis treated with posterior lumbar interbody fusion using BAK cages. Spine, 30(19), 2171-2175. See https://doi.org/10.1097/01.brs.0000180402.50500.5b
2. Kimura, H., Shikata, J., Odate, S., Soeda, T., & Yamamura, S. (2012). Risk factors for cage retropulsion after posterior lumbar interbody fusion: analysis of 1070 cases. Spine, 37(13), 1164-1169. See https://doi.org/10.1097/BRS.0b013e318257f12a
3. Lin, G. X., Kotheeranurak, V., Zeng, T. H., Mahatthanatrakul, A., & Kim, J. S. (2019). A longitudinal investigation of the endplate cystic lesion effect on oblique lumbar interbody fusion. Clinical neurology and neurosurgery, 184, 105407. See https://doi.org/10.1016/j.clineuro.2019.105407
4. Malham, G. M., Parker, R. M., Blecher, C. M., & See, K. A. (2015). Assessment and classification of subsidence after lateral interbody fusion using serial computed tomography. Journal of neurosurgery. Spine, 23(5), 589-597. See https://doi.org/10.3171/2015.1.SPINE14566
5. Nemani, V. M., Aichmair, A., Taher, F., Lebl, D. R., Hughes, A. P., Sama, A. A., Cammisa, F. P., & Girardi, F. P. (2014). Rate of revision surgery after stand-alone lateral lumbar interbody fusion for lumbar spinal stenosis. Spine, 39(5), E326-E331. See https://doi.org/10.1097/BRS.0000000000000141
6. Hou, Y., Shi, H., Shi, H., Zhao, T., Shi, J., & Shi, G. (2023). A meta-analysis of risk factors for cage migration after lumbar fusion surgery. World neurosurgery: X, 18, 100152. See https://doi.org/10.1016/j.wnsx.2023.100152
7. Park, M. K., Kim, K. T., Bang, W. S., Cho, D. C., Sung, J. K., Lee, Y. S., Lee, C. K., Kim, C. H., Kwon, B. K., Lee, W. K., & Han, I. (2019). Risk factors for cage migration and cage retropulsion following transforaminal lumbar interbody fusion. The spine journal : official journal of the North American Spine Society, 19(3), 437-447. See https://doi.org/10.1016/j.spinee.2018.08.007
8. Park, J. Y., Choi, K. Y., Moon, B. J., Hur, H., Jang, J. W., & Lee, J. K. (2016). Subsidence after single-level anterior cervical fusion with a stand-alone cage. Journal of clinical neuroscience : official journal of the Neurosurgical Society of Australasia, 33, 83-88. https://doi.org/10.1016/j.jocn.2016.01.042
9. Maccormick, A. P., & Sharma, H. (2020). Analysis of the Variables Affecting the Incidence, Location, and Severity of Cage Subsidence Following Anterior Cervical Discectomy and Fusion Operation. International journal of spine surgery, 14(6), 896-900. See https://doi.org/10.14444/7137
10. Lang, G., Navarro-Ramirez, R., Gandevia, L., Hussain, I., Nakhla, J., Zubkov, M., & Härtl, R. (2017). Elimination of Subsidence with 26-mm-Wide Cages in Extreme Lateral Interbody Fusion. World neurosurgery, 104, 644-652. See https://doi.org/10.1016/j.wneu.2017.05.035
11. Teng, I., Han, J., Phan, K., & Mobbs, R. (2017). A meta-analysis comparing ALIF, PLIF, TLIF and LLIF. Journal of clinical neuroscience : official journal of the Neurosurgical Society of Australasia, 44, 11-17. See https://doi.org/10.1016/j.jocn.2017.06.013
12. Yang, J.H., Tuano, K.R., Finn, M. et al. Successful repair of chronic esophageal perforation due to hardware migration with buttressing free gracilis flap: a case report. Eur J Plast Surg 45, 983-987 (2022). See https://doi.org/10.1007/s00238-021-01936-9
13. Prusick, P. J., Sabri, S. A., & Kleck, C. J. (2021). Expectoration of anterior cervical discectomy and fusion cage: a case report. Journal of spine surgery (Hong Kong), 7(2), 218-224. See https://doi.org/10.21037/jss-20-655
14. Korovessis, P., Repantis, T., Vitsas, V., & Vardakastanis, K. (2013). Cervical spondylodiscitis associated with oesophageal perforation: a rare complication after anterior cervical fusion. European journal of orthopaedic surgery & traumatology : orthopedie traumatologie, 23 Suppl 2, S159-S163. See https://doi.org/10.1007/s00590-012-1092-y

1. An expandable intervertebral device for insertion between two vertebral bodies to reestablish the appropriate space and angle of the spine, comprising:
a central component having proximal and distal central pieces with superior molded angled interlocks and also with inferior molded angled interlocks, the proximal and distal central pieces being configured to slide transversely relative to each other;
a superior plate having proximal and distal ends with molded angled interlocks configured to connect to the superior molded angled interlocks of the proximal and distal central pieces and allow for uninhibited sliding of the superior plate relative to the proximal and distal central pieces;
an inferior plate having corresponding proximal and distal ends with corresponding molded angled interlocks configured to connect to the inferior molded angled interlocks of the proximal and distal central pieces and allow for uninhibited sliding of the inferior plate relative to the proximal and distal central central pieces; and
a dual-access screw configured to connect the proximal and distal central pieces and modulate transverse compression and expansion of the proximal and distal central pieces and vertical expansion and compression of the superior plate and the inferior plate respectively.

2. The expandable intervertebral device according to claim 1, wherein the expandable intervertebral device comprises one or more cannulated channels configured to pass through the expandable intervertebral device and receive high-strength suture to be threated through and subsequent fixation to adjacent osseus structures via bone anchors.

3. The expandable intervertebral device according to claim 1, wherein the proximal and distal central pieces each have a respective cannulated opening configured for the dual-access screw to be threaded through.

4. The expandable intervertebral device according to claim 3, wherein the dual-access screw has a head; and the respective cannulated opening in a proximal central piece of the proximal and distal central pieces has a diameter that is greater than a corresponding diameter where the head of the dual-access screw sits.

5. The expandable intervertebral device according to claim 3, wherein the dual-access screw has a headless portion and a body having threads with an inner and outer diameter; and a distal central piece of the proximal and distal central pieces has a cannulated portion that is threaded and matches the inner and outer diameter of the threads of the body of the dual-access screw.

6. The expandable intervertebral device according to claim 1, wherein the dual-access screw includes a proximal end and a headless distal end, and the proximal end has a proximal head.

7. A method for fixation and removal of the expandable intervertebral device according to claim 1, wherein the method comprises:
modulating with the dual-access screw vertical expansion and compression of the superior plate and the inferior plate by either transverse compression or expansion of proximal and distal central pieces respectively;
integrating the one or more cannulated channels within the expandable intervertebral device with one or more high-strength sutures for direct fixation via the one or more associated bone anchors; and
fixing externally the one or more high-strength sutures with the one or more associated bone anchors to surrounding osseus structures for indirect buttressing of the expandable intervertebral device.

8. The method according to claim 7, wherein the method comprises rotating a proximal head of a proximal end of the dual-access screw clockwise, or a headless distal end counterclockwise for vertical expansion of the superior plate and the inferior plate.

9. The method according to claim 7, wherein the method comprises rotating a proximal head of a proximal end of the dual-access screw counterclockwise, or rotating a headless distal end clockwise for vertical compression of the superior plate and the inferior plate.

10. The method according to claim 7, wherein the method comprises fixing the one or more high-strength sutures at either one or more ends of the expandable intervertebral device.

11. The method according to claim 7, wherein the method comprises either integrating the one or more high-strength sutures into the one or more cannulated channels of the expandable intervertebral device, or using the one or more high-strength sutures external to the expandable intervertebral device as a low-profile buttress.

12. The method according to claim 7, wherein the method comprises fixing the one or more high-strength sutures via the one or more associated bone anchors in vertical and/or crossing latticework patterns.

13. The method according to claim 7, wherein the method comprises adapting the expandable intervertebral device to an insertion handle for implantation and subsequent bone graft integration for intervertebral unionization.

14. The method according to claim 7, wherein the method is implemented in a revision surgery using a similar or different approach as is used in a primary surgery by exposing ends of the expandable intervertebral device for access to one of a proximal or distal end of the dual-access screw for removal of the expandable intervertebral device and insertion of a new intervertebral implant.

15. A method for inserting an expandable intervertebral device between two vertebral bodies to reestablish the appropriate space and angle of the spine, comprising:
configuring a central component having proximal and distal central pieces with superior molded angled interlocks and also with inferior molded angled interlocks to slide transversely relative to each other;
configuring a superior plate having proximal and distal ends with molded angled interlocks to connect to the superior molded angled interlocks of the proximal and distal central pieces and allow for uninhibited sliding of the superior plate relative to the proximal and distal central pieces;
configuring an inferior plate having corresponding proximal and distal ends with corresponding molded angled interlocks to connect to the inferior molded angled interlocks of the proximal and distal central pieces and allow for uninhibited sliding of the inferior plate relative to the proximal and distal central pieces; and
connecting with a dual-access screw the proximal and distal central pieces and modulating transverse compression and expansion of the proximal and distal central pieces and vertical expansion and compression of the superior plate and the inferior plate respectively.

16. The method according to claim 15, wherein the method further comprises configuring one or more cannulated channels to pass through the expandable intervertebral device and receive one or more high-strength sutures to be threated through for subsequent fixation to adjacent osseus structures via one or more associated bone anchors.

17. The method according to claim 15, wherein the method comprises configuring each of the proximal and distal central pieces with a respective cannulated opening for threading the dual-access screw through.

18. The method according to claim 15, wherein the method comprises configuring the dual-access screw with a proximal end and a headless distal end, and the proximal end with a proximal head.

19. The method according to claim 15, wherein the method comprises configuring the dual-access screw with a head; and configuring the respective cannulated opening in a proximal central piece of the proximal and distal central pieces with a diameter that is greater than a corresponding diameter where the head of the dual-access screw sits.

20. The method according to claim 15, wherein the method comprises configuring the dual-access screw with a headless portion and a body having threads with an inner and outer diameter; and configuring a distal central piece of the proximal and distal central pieces with a cannulated portion that is threaded and matches the inner and outer diameter of the threads of the body and the headless portion of the dual-access screw.
